(19) United States
(12) Patent Application Publication
(10) Pub. No.: US 2019/0213338 A1
(43) Pub. Date: Jul. 11, 2019

(54) METHOD FOR VERIFYING THE INTEGRITY OF AN ADDITIVE MANUFACTURING PROCESS

(71) Applicants: Rutgers, The State University of New Jersey, New Brunswick, NJ (US); Georgia Tech Research Corporation, Atlanta, GA (US)

(72) Inventors: Saman Zonouz, New Brunswick, NJ (US); Mehdi Javanmard, West Windsor, NJ (US); Raheem Beyah, Atlanta, GA (US); Luis A. Garcia, Somerset, NJ (US); Tuan-Anh Le, Manville, NJ (US); Christian Bayens, Atlanta, GA (US)

(21) Appl. No.: 16/244,632

(22) Filed: Jan. 10, 2019

Related U.S. Application Data
(60) Provisional application No. 62/615,669, filed on Jan. 10, 2018.

(51) Int. Cl. G06F 21/60; B29C 64/393; G06F 17/50; B29C 64/209

(52) U.S. Cl. CPC G06F 21/608; B29C 64/393; B33Y 50/02; B29C 64/209; G06F 17/50

(57) ABSTRACT

This disclosure provides methods for verifying the integrity of an additive manufacturing process during or after a three-dimensional (3D) print job. The methods include at least one of three validation layers: an acoustic layer, a spatial sensing layer, and a material verification layer. For the acoustic layer, the method includes determining the presence of a signature audio signal. For the spatial sensing layer, the method includes comparing a recorded trajectory with a reference trajectory. The method also includes determining the presence of a signature trajectory. For the material verification layer, the method includes determining the location of a special material in a 3D printed object based on a predetermined pattern in which the special material embedded in a filament. The methods allow for detecting alteration in the additive manufacturing process.

### CROSS-REFERENCE TO RELATED APPLICATIONS

This patent document claims priority under 35 U.S.C. § 119(e) to the U.S. Provisional Patent Application No. 62/615,669, filed Jan. 10, 2018. The patent application identified above is incorporated here by reference in its entirety to provide continuity of disclosure.

### STATEMENT REGARDING FEDERALLY-SPONSORED RESEARCH

This invention was made with government support under Grant No. 1534872 awarded by the National Science Foundation. The government has certain rights in the invention.

### FIELD OF THE INVENTION

The present invention generally relates to additive manufacturing and more particularly to methods for verifying the integrity of an additive manufacturing process.

### BACKGROUND OF THE INVENTION

Additive Manufacturing (AM) is an increasingly integral part of industrial manufacturing. Safety-critical products, such as medical prostheses and parts for aerospace and automotive industries, are being printed by additive manufacturing methods with no standard means of verification. Additive Manufacturing, also known as 3D printing, is an emerging field that shows promise in reducing waste, time, and infrastructure needed in a manufacturing process. Many major companies including Ford, GE, Airbus, SpaceX, Koenigsegg, and NASA are currently utilizing AM for both prototyping and production-quality manufacturing. Additionally, AM is a useful tool for printing medical implants, and cutting-edge research is underway on producing food, drugs, and living tissue using AM techniques. Across industries, AM is expected to reach a market potential of 50% by 2038.

Because of this potential for wide-spread use of AM in the coming decades, work has begun on understanding the security challenges that are unique to traditional manufacturing and cyber-physical security. Yampolskiy et al., outlined a taxonomy for the potential of the misuse of a 3D printer as a weapon (3D-PaaW) and identified the elements which may compromise or manipulate an AM environment, the targets of attack (printed object, printers, or environment), and the parameters for understanding the potential effectiveness of a given attack (Yampolskiy, et al. Int. J. Crit. Infrastruct. Prot. 14, C (September 2016), 58-71).

Kinetic Cyber-Attack Detection Method (KCAD) provided the first method of using the analog emissions of AM processes to detect so-called zero-day kinetic cyber-attacks. However, the work utilizes only one 3D printer and only investigates attacks in which simple variations in the exterior design. It also lacks any means of verifying the printed materials post-manufacturing. It has been demonstrated that the array of sensors available on a modern smartphone can be leveraged to re-create designs produced by 3D printers or CNC machines. The sensors used in each study to collect side-channel data included the microphone, magnetometer, and accelerometer. Each group was able to reconstruct simple printed designs using supervised machine learning and manual analysis of sensor signals respectively. However, each group was only able to reconstruct very simple shapes such as two-dimensional outlines of airplanes or keys with no fill structure.

The physical model printed from the AM machines is typically verified in a manner specific to the domain, such as mechanical strength testing. Chien et al., use several techniques such as surface morphology characterization to verify 3D-printed tissue scaffolds (Chien et al., Tissue Engineering Part C: Methods, 19(6):417-426; 2012). Furthermore, several solutions have been presented as preventative measures to future physical failures, such as the solution presented by Stava, et al., for detecting and correcting models prior to being printed (Stava, et al., ACM Transactions on Graphics (TOG), 31(4):48, 2012). However, these only correct the models that are being sent to the printer and do not verify the actual physical model in the event that the printer itself is compromised.

Thus, a strong need in the art exists for a method of monitoring the integrity of the additive manufacturing process during the course of a print and verifying the integrity of the print after production.

### SUMMARY OF THE INVENTION

The present invention addresses this need by providing a method for verifying the integrity of an additive manufacturing process. The method may include: (1) printing a three-dimensional (3D) object using a filament. The filament includes a first material and a second material. The second material is embedded in the first material in a predetermined pattern; (2) determining a reference location of the second material in the 3D object based on the predetermined pattern of the second material in the filament; (3) detecting a target location of the second material in the 3D object; (4) calculating a difference between the reference location and the target location; and (5) determining the presence of alteration of the additive manufacturing process if the difference between the reference location and the target location is above a threshold value.

In some embodiments, the second material may include a nanoparticle, a gold nanorod (GNR), or 3, 3′-Diethylthiatricarbocyanine iodide (DTTCI). In some embodiments, the predetermined pattern is encoded in a barcode. In determining the reference location of the second material inside the 3D object, the method may include decoding the barcode to extract the predetermined pattern of the second material.

The target location of the second material in the object may be determined using an imaging tool, such as X-ray, IR, Raman, or computed tomography.

In another aspect, the method may include: (1) recording, by an audio sensor (e.g., wide-range microphone, directional microphone), an audio signal generated by the 3D printer during the performance of the 3D print job; (2) comparing, by a computer, the recorded audio signal with a reference recording during or after the performance of the 3D print job; (3) determining a difference between the recorded audio signal and the reference recording; and (4) determining that the additive manufacturing process is altered if the difference is below a threshold value (e.g., the confidence threshold (CTh)).

In some embodiments, each of the audio signal and the reference recording includes a plurality of audio segments (e.g., 90-second segments, 120-second segments). The method may also comprise determining a difference between an audio segment of the audio and a corresponding audio segment of the reference recording. The method may further comprise determining that the additive manufacturing process is altered if the difference is below the threshold value.

In another aspect, the method may include recording, by a sensor (e.g., gyroscopic sensor, linear potentiometer), a trajectory of a printer head of a three-dimensional (3D) printer during a performance of a 3D print job; and determining a presence of alteration of an additive manufacturing process associated with the performance of the 3D print job based on the recorded trajectory. In some embodiments, the method may further include comparing, by a computer, the recorded trajectory with a reference recording during or after the performance of the 3D print job; and determining the presence of the alteration of an additive manufacturing process associated with the performance of the 3D print job based on a difference between the recording trajectory and the reference recording.

In some embodiments, the method may include (1) generating instructions for causing the printer head to produce a signature trajectory during the performance of the 3D print job; (2) determining, by the computer, whether the signature trajectory is present in the recorded trajectory; and (3) determining that the additive manufacturing process associated with the performance of the 3D print job is altered if the signature trajectory is absent in the recorded trajectory.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows a system model that includes verification techniques employed in the present invention. The design will be printed on a 3D printer that is controlled by a controller PC. The 3D printer may or may not be controlled by a third party entity. The end user may send the design to be printed. Throughout the printing process, the object may be verified using at least one of three verification layers. The first two layers are achieved through acoustic side-channel analysis and spatial sensing which analyze the sound and physical position of printing components, respectively. The third layer is that of materials verification in which imaging techniques are used to verify that the print is made from the proper material and printed correctly.

FIG. 2A and FIG. 2B show 3D printed models described as a top hat (FIG. 2A) and a rectangular prism (FIG. 2B).

FIG. 3A and FIG. 3B (collectively “FIG. 3”) show exemplary classification results.

FIG. 4 shows a comparison of G-code reconstruction to gyroscopic sensing reconstruction of single layers of various fill types and densities.

FIG. 5 is Raman scattering measurement of a silicon wafer with gold nanorods (GNRs) and 3, 3′-Diethylthiatricarbocyanine iodide (DTTCI). The Raman spectrum of Si is amplified when using the enhancers.

FIG. 6A and FIG. 6B (collectively “FIG. 6”) are computed tomography (CT) scans of acrylonitrile butadiene styrene (ABS) control print (FIG. 6A) and ABS cylindrical tube with embedded GNRs (FIG. 6B).

FIG. 7A shows Receiver Operating Characteristic (ROC) curves for the rectangular prism with a confidence threshold (CTh) of 35; FIG. 7B shows ROC curves for top hat; FIG. 7C is a comparison of the frequency response between a single layer of honeycomb 40% fill and rectilinear 40% fill. Four samples of each fill are compared; FIG. 7D shows ROC curves for the top hat design printed using a TazMini, Orion Delta, and Taz6 print. Prints audio was sliced to 120 seconds, and CTh is 150, 20, and 35, respectively.

FIG. 8 shows mean measurement of Raman scattering of 3D printed disks using ABS filament and ABS embedded with GNRs and 3,3′-Diethylthiatricarbocyanine iodide (DTTCI).

FIG. 9A and FIG. 9B (collectively “FIG. 9”) show a comparison between target 60% rectilinear fill tibial prosthetic print acoustic classification (FIG. 9A) and malicious 20% honeycomb fill (FIG. 9B) with CTh=0.

FIG. 10 is a comparison of x-axis frequency response for a layer of the tibial knee implant design.

FIG. 11 is a comparison of target and malicious tibial knee implant prints. Left: G-code reconstruction of 60% rectilinear fill, Middle: Spatial reconstruction of 60% rectilinear fill, Right: Spatial reconstruction of malicious 20% honeycomb fill.

FIG. 12A shows an X-ray scan of the front of polylactic acid (PLA) tibia with embedded stainless steel at a 15 μm/voxel size resolution. The first label (label 1) shows the side view of the cross-sectional stainless steel infill, while the second label (label 2) shows the two blotches where the stainless steel print began; FIG. 12B and FIG. 12C is a comparison of G-code simulation of embedded steel (FIG. 12B) versus CT scan of the printed model (FIG. 12C). The CT scan image is rotated about 45 degrees.

### DETAILED DESCRIPTION OF THE INVENTION

The present invention is directed to a method for verifying the integrity of an additive manufacturing process associated with a 3D print job. The method may include printing a 3D object using a filament which may include a first material and a second material. The second material may be embedded in the first material in a predetermined pattern. The first material and the second material can be any of common materials for 3D printing, including but not limited to, plastics (e.g., nylon or polyamide, ABS, PLA, TPU), charged plastics (e.g., alumide, carbonmide), metal (e.g., stainless steel, titanium, aluminum, brass, silver), and resin (e.g., polyjet resins, CLIP resins including elastomeric polyurethane (EPU), rigid polyurethane (RPU), cyanate ester (CE), flexible polyurethane (FPU), epoxy resin). The second material of the filament may include one or more materials which allow detection of a location of the second material in a 3D printed object. For example, the second material of the filament may be a nanoparticle or a contrast agent, such as gold nanorod or 3,3′-Diethylthiatricarbocyanine iodide. The filament mentioned above may be provided by the end user to the 3D printing service provider, so that the end user can perform physical model verification upon completion. The end user may also provide a modified filament embedded with a special material. The modified filament can be used for materials verification purposes.

The second material is embedded in the first material in a predetermined pattern. The predetermined pattern may be defined by the location in the filament where the second material is embedded and/or the space between the embedded locations. The information specific to the second material itself and the predetermined pattern may be encoded in a barcode, which can be indexed and stored in a database. Combined with instructions of a 3D printing design in stereolithography (STL) files, such information can be used to determine a reference location of the second material in a 3D printed object. Determination of the reference location of the second material inside the 3D object may include decoding the barcode to extract the predetermined pattern of the second material from the database. A detection method may be used to determine a target location of the second material in the 3D printed object. The detection method may be an imaging tool, such as X-ray, an IR, a Raman, or a computed tomography.

The method for verifying the integrity of an additive manufacturing process may include determining a reference location of the second material in the 3D object based on the predetermined pattern in the filament and detecting a target location of the second material in the 3D object. The method may further include determining a presence of alteration of the additive manufacturing process if the calculated difference between the reference location and the target location is above a threshold value, e.g., a confidence threshold (CTh) value.

This disclosure also provides a method for verifying the integrity of an additive manufacturing process by: (i) generating instructions to cause a 3D printer to produce a signature sound during the performance of a 3D print job; and (ii) recording an audio signal generated by the 3D printer using an audio sensor. The instructions may be included in an STL file or a G-code file. The instructions may cause a printer head and/or other components of the 3D printer to carry out movements resulting in the signature sound. The audio sensor may be a microphone which records the audio signal during the printing process. The audio signal may be transferred to a storage medium and processed into a digital format for analysis.

The method may also include determining a presence of the signature sound in the recorded audio signal by comparing the recorded audio signal with a reference recording during or after the performance of the 3D print job. The method may further include determining the presence of alteration of the additive manufacturing process based on the presence of the signature sound in the recorded audio signal. The audio signal and the reference recording each may comprise a plurality of audio segments. The method may include comparing a segment of the audio with a corresponding segment of the reference recording.

This disclosure also provides a method for verifying the integrity of an additive manufacturing process, which may include recording a trajectory of a component of a 3D printer during a 3D print job by a sensor and determining a presence of alteration of the additive manufacturing process associated with the 3D print job based on the recorded trajectory. The component of the 3D printer may be a printer head or a supporting arm connected to the printer head. The sensor used to record the trajectory of the component of the 3D printer may include a gyroscopic sensor and a linear potentiometer. The recorded trajectory of a 3D printed object may be compared with a reference trajectory recorded for a reference object. The presence of alteration of the additive manufacturing process may be confirmed if the difference between the recorded trajectory and the reference trajectory is above a threshold value.

The method may further include generating instructions for causing a component of a 3D printer to produce a signature trajectory during the performance of the 3D print job. The component of the 3D printer may be a printer head or a supporting arm connected to the printer head. The method may include determining whether the additive manufacturing process is altered based on the presence of the signature trajectory in the recorded trajectory.

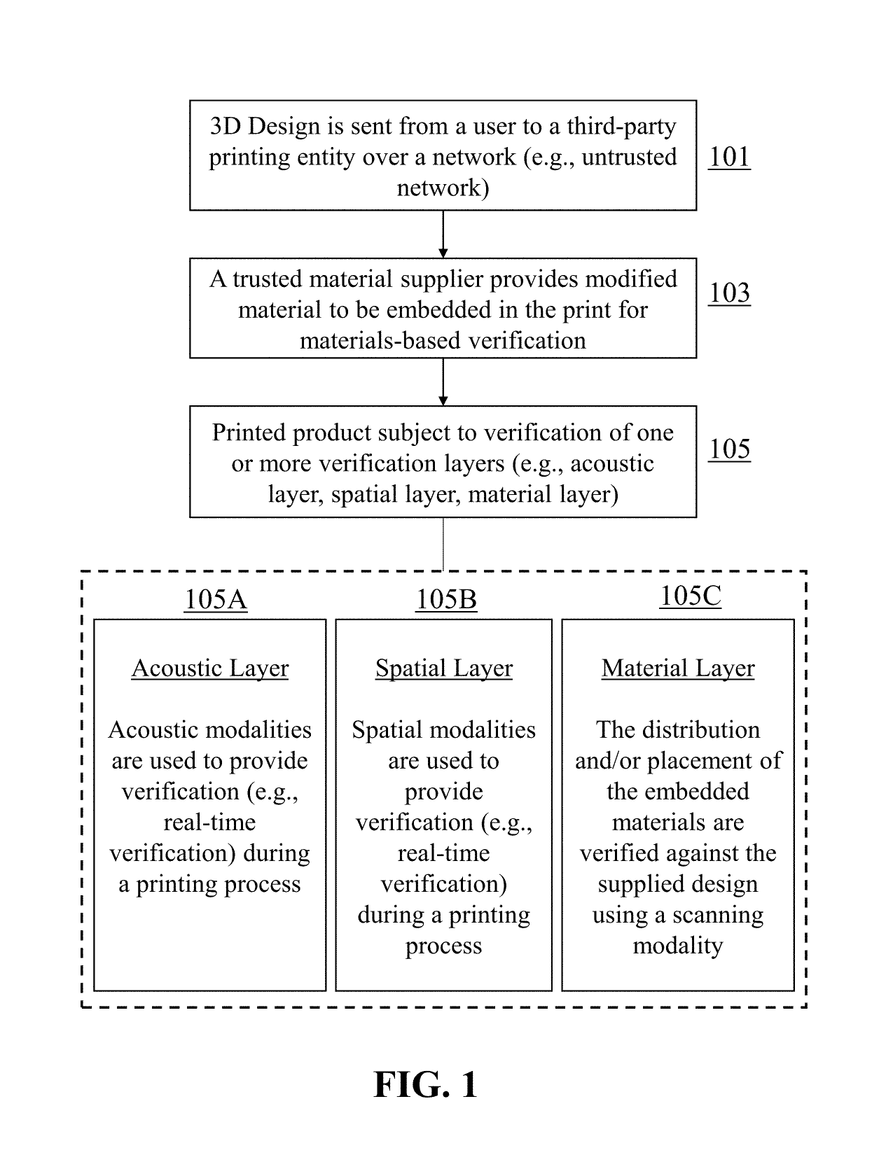

Referring now to FIG. 1, an overview of a system model that includes verification techniques is provided. At 101, a user may send a 3D design to a third-party printing entity over a network. The 3D design may be any of file formats known in the art, include without limitation, 3D Studio Max (.max, 0.3ds), AC3D (.AC), Apple 3DMF (0.3dm/0.3dmf), Autocad (.dwg), Blender (.blend), Caligari Object (.cob), Collada (.dae), Dassault (0.3dxml), DEC Object File Format (.off), DirectX 3D Model (.x), Drawing Interchange Format (.dxf), DXF Extensible 3D (.x3d), Form-Z (.fmz), GameExchange2-Mirai (.gof), Google Earth (.kml/.kmz), HOOPS HSF (.hsf), LightWave (.lwo/.lws), Lightwave Motion (.mot), MicroStation (.dgn), Nendo (.ndo), OBJ (.obj), Okino Transfer File Format (.bdf), OpenFlight (.flt), Openinventor (.iv), Pro Engineer (.slp), Radiosity (.radio), Raw Faces (.raw), RenderWare Object (.rwx), Revit (.rvt), Sketchup (.skp), Softimage XSI (.xsi), Stanford PLY (.ply), STEP (.stp), Stereo Litography (.stl), Strata StudioPro (.vis), TrueSpace (.cob), trueSpace (.cob, .scn), Universal (.u3d), VectorWorks (.mcd), VideoScape (.obj), Viewpoint (.vet), VRML (.wrl), Wavefront (.obj), Wings 3D (.wings), X3D Extensible 3D (.x3d), Xfig Export (.fig).

The network may include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network. In some examples, the network is an untrusted network which posts risks that the 3D design could be intercepted or tampered during the transmitting process.

The design supplied by an end user can be printed on a 3D printer controlled by a controller PC. The 3D printer may or may not be controlled by a third party entity. At 103, the third party printing entity may use modified material provided by a trusted material supplier. The modified material can be embedded in the print for materials-based verification. Throughout the printing process, the object can be verified using one or more of three verification layers 105. The first two layers can be achieved through acoustic side-channel analysis 105A and spatial sensing 105B which analyze the sound and physical position of printing components, respectively. The third layer is a material verification layer 105C in which imaging techniques may be used to verify that the print is made from a proper material and printed correctly.

The threat model assumes that the attacker has full knowledge of both the printer and its control software. If a third party manufacturer or affiliate of the user is involved, they are trusted as an organization. Therefore, they are willing to provide information about the print for verification. However, malicious entities may include network intruders, disgruntled employees, or other insider threats. The attack is carried out such that the printer behaves maliciously despite being sent G-code for a non-malicious print. G-code is the set of instructions that can be interpreted by a 3D printer or CNC and includes information about motion direction, speed, and other operations. Meanwhile, the controller PC indicates that the print is being carried out correctly. This attack feasible using a cyber-physical rootkit has been described previously.

It is also assumed that training prints may be performed under supervised circumstances in which it may be reasonably assumed that no attack is taking place. This may be achieved by a direct connection between the controlling machine and the printer via USB. The materials supplier, as shown in FIG. 1, is assumed to be trusted. Untrusted materials suppliers are beyond the scope of this paper. For the materials-based verification, the modified filaments with the embedded materials are to be supplied directly by the end user. Furthermore, all communication channels among trusted entities are assumed to be secure.

### EXAMPLES

### Example 1

Verification Layers and Implementation

It is important to verify the internal fill structure present in all 3D printed objects. When a print is converted from a design to G-code instructions for a 3D printer or a computer numerical control (CNC) machine, an internal structure for the physical product must be generated. It ranges from low density for prototyping or non-load bearing prints to high density for load bearing or industrial use. The fill itself may have a honeycomb pattern, rectilinear pattern, or other types of grids as specified by the user. Failure to produce the proper internal fill results in a final printed product that looks like the intended design externally, but fails to provide desired physical characteristics.

To develop a robust verification scheme, methods are needed to allow for (i) real-time identification; (2) visualization of potentially malicious prints; and (3) visualization of a completed print to ensure its usability. Analysis of the acoustic side-channel was used as a non-intrusive method of identification. In addition, for real-time visualization, tracking the moving components of a printer or CNC machine was shown to be a useful way of understanding the process without relying on the control software.

### 1.1. Side-Channel Verification

The side-channel analysis verification layers provide a means of verifying printed models in real-time. The goal will be to infer as much information as possible from the given side-channels, with each modality contributes to the verification of the entire print. The experimental design for each side-channel modality is described below.

### Acoustic Layer

As a physical byproduct of nearly any mechanical process, acoustic signals have been explored as a method of understanding information being processed by both traditional printers and 3D printers used in AM. Because traditional printing methods now rely on lasers or inkjets, the information obtained from these is minimal. In contrast, 3D printers will continue to rely on various actuators and fans for the foreseeable future which produce useful acoustic data. This is especially true for large-scale implementations of the technology.

In this verification layer, it is assumed that a particular design with a given infill structure will be printed multiple times. An open source audio classifier similar to the Shazaam or SoundHound applications is used (Wang et al., ISMIR, 2003). Using a training audio file, it locates noise-resistant peak frequencies and their temporal location within the file. It then creates a hash of the peak information and looks for collisions in test data. When a test file is identified, it is accompanied by a confidence score among other information. The confidence score indicates the number of peaks that the test has in common with the training data.

For AM verification, a single print is used as a training set by recording it with a microphone to obtain an audio file. Because even a simple print can take many minutes, if necessary, the resulting file is segmented into a number segments of a given length (e.g., 10 seconds, 30 seconds, 60 seconds) and indexed in ascending order. Each indexed segment of the print is then trained as a different “song” and stored in a database. Compared to typical machine learning schema where common practice is to train multiple sets of data, the acoustic classification involves one-to-one comparison of audio files, and thus a single-file training set is more appropriate.

Test data were collected using the same method as training data and split into equal length segments. Each indexed segment was then classified independently, and a confidence score was returned. The confidence score represents the number of frequency peaks that a given file has with the training file. There are two ways to verify from the training set that a repeated print is unaltered: (1) the classification results having the index values that appear in ascending order. If they are out of order, it is likely that a change has been made; and (2) the confidence score of one or more indexed classification results falls below a given threshold value (also referred to as the confidence threshold (CTh)). CTh was optimized to maximize the true positive rate and minimize the false positive rate. A print will be considered verified if each indexed audio file being classified correctly, in the correct order, and with confidence values greater than the CTh. Conversely, a non-verified print will be classified but out of order or with one or more confidence values less than CTh.

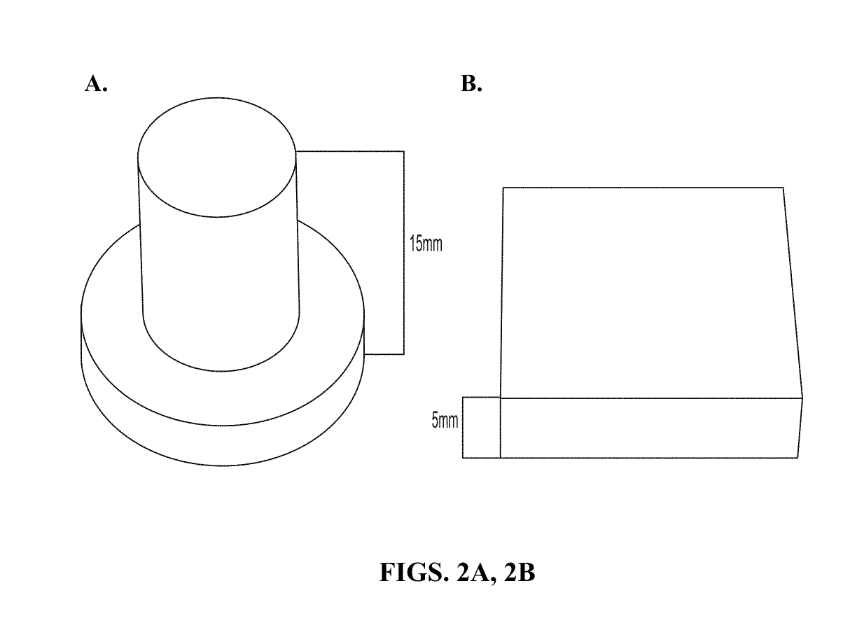

To test this method, two designs, as shown in FIG. 2, described as a rectangular prism (FIG. 2B) and a top hat (FIG. 2A) were tested throughout this section. Each was printed several times with “honeycomb” and “rectilinear” fill patterns of 20%, 40%, and 60% density. For each print style, a single set of audio data was split and stored in a database described above.

To produce quantitative results of the test classifications, a “score” was assigned to each segment of the audio data which are defined as follows: (i) if a segment is in proper sequence, and the confidence value is greater than CTh, its score is equal to that of the confidence value; (ii) if a segment is out of sequence, its score is equal to −1× confidence value; and (iii) if a segment is in sequence, but the confidence value is less than CTh, its score is set equal to −1× confidence value. If any segment of the audio file is associated with a negative score, a positive error classification may be determined. Otherwise, a negative error classification of the audio file is determined.

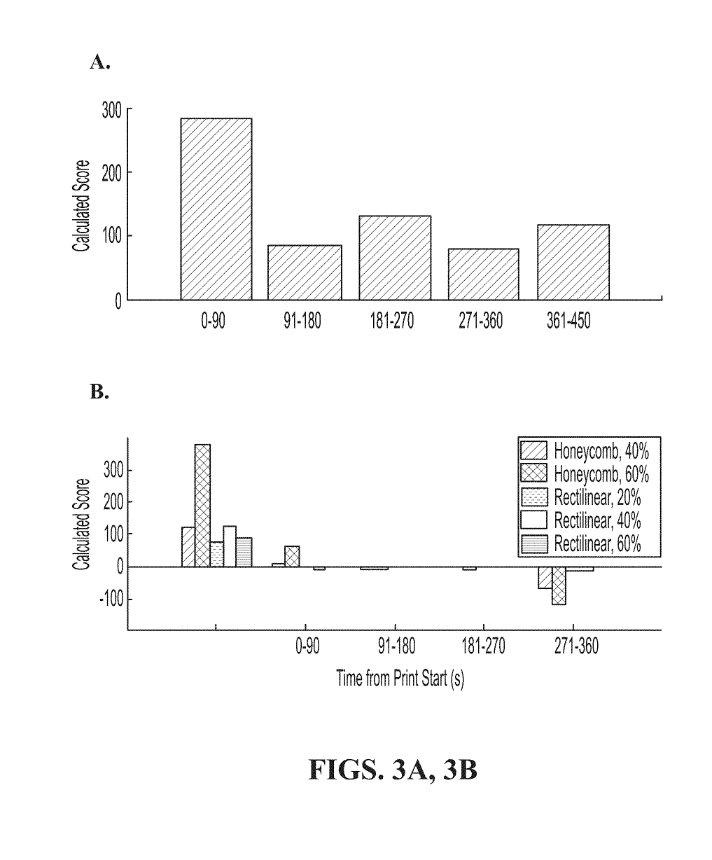

FIG. 3A and FIG. 3B provide sample results of the print of a rectangular prism with a 20% density honeycomb fill pattern. FIG. 3A shows the averaged results of three known negative error classifications (true negatives). Each bar represents a 90-second slice of the printing data, and CTh was set to 35. Likewise, FIG. 3B represents various positive error classifications (true positives) caused by incorrect fill densities or patterns. Each type of error was printed four times, and the results were averaged. For errors involving the honeycomb fill pattern with erroneous densities, a positive error classification was achieved within 270 s or the first 60% of the print. For the erroneous rectilinear fill pattern, positive error classification was achieved within 180 s or 40% of the print. In each case, the first 90 s slice always receives high scores due to the fact that the design always starts with a 100% rectilinear fill of the first three layers. This is standard in 3D printing to ensure that the exterior is solid.

### Spatial Sensing Layer

When performing 3D prints, the software that monitors print progress typically only displays the progress of the G-code instructions being sent to the printer regardless of the actual actions of the printer. Thus, the goal of the spatial sensing verification scheme was to physically monitor the position of one or more printer parts (e.g., printing nozzle) with respect to the printing base to determine their actual positions throughout the printing process.

The first consideration was to use a ride-along accelerator. However, due to the double integration from acceleration to position and the noisiness of the accelerometer data, visual representations of the printer's path became prohibitively difficult to obtain. With this in mind, a scheme was developed in which a gyroscopic sensor was paired with a linear potentiometer in order to construct a set of spherical coordinates to describe the printer's motion. This proved more effective because no integration was needed for the data, and only simple moving average filtering was necessary to reduce noise.

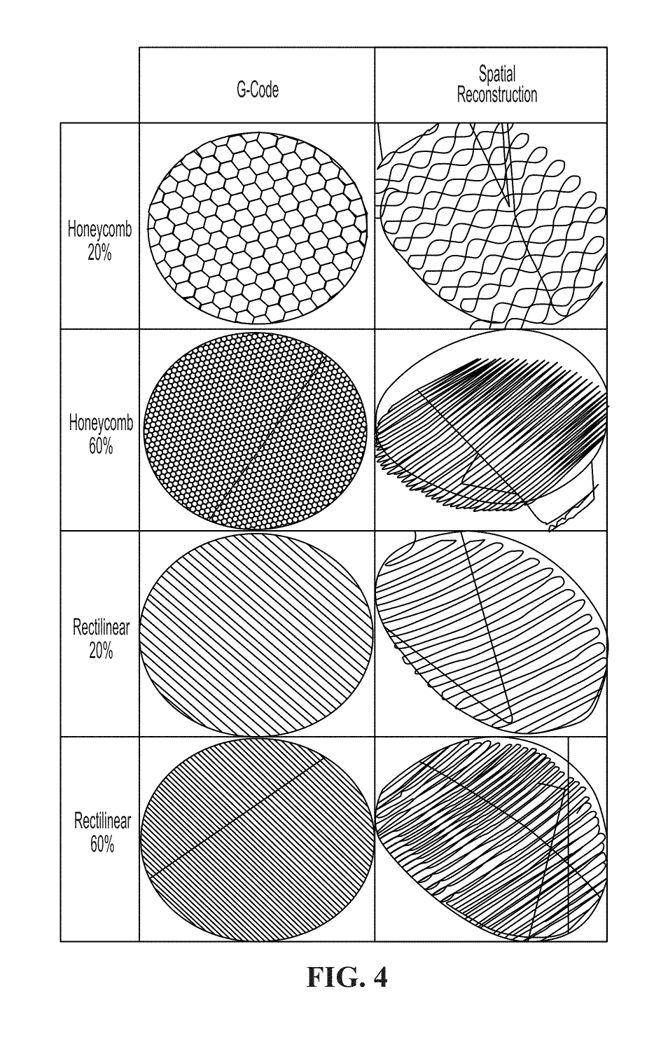

To obtain these measurements, the following devices were used: a Unimeasure linear potentiometer model number LA-PA-10-N1N-NPC, a SparkFun Triple Axis Accelerometer and Gyro Breakout MPU-6050, and a Teensy 3.2 board. The experiments were conducted in a setup with a Dobot Magician desktop CNC and 3D Printer. For experimental purposes, the actual 3D printing extruder was removed, and “dummy” prints were performed. The test prints were a single layer of a circular disk printed with honeycomb and rectangular fills each with a 20% and 60% density (FIG. 4). Data were collected at a rate of 100 Hz. Each print was shown as the G-code representation next to the reconstructed path of the printer. The data were smoothed using a moving average filter with a window of five.

### 1.2. Material Verification

The objective of the materials-based verification is to embed contrast agents that will act as signature markers for particular prints without compromising the structural integrity of the original model. The contrast agents were chosen based on the materials as well as the scanning modalities. A single type of nanoparticle was embedded at different points in the printed model to generate a pattern specific to the model. This ensures that the model was not modified by either an attacker who compromised the firmware and who is duping the manufacturer or a malicious insider who has physical access to the printing process. While it is arguable that embedded markers would change the integrity of the material itself, numerous studies have shown that the use of nanoparticles actually improves the materials' mechanical strength.

In this study, two types of scanning modalities have been explored: Raman spectroscopy and computed tomography (CT). The goal was to explore their effectiveness in the disclosed verification techniques. In both cases, it is assumed that the end user will provide the necessary materials to the manufacturer, who will be responsible for printing the model. The design sent to the manufacturer will not include any information about the embedded materials.

### Raman Spectroscopy

Raman spectroscopy has been shown to be useful for specific target identification and quantification. The target sample was irradiated with a monochromatic light source, such as a laser. The majority of the scattering light has the same frequency of the incident light. This elastic scattering is called Rayleigh scattering. A small fraction of the scattering is inelastic. It has a small shift in photon frequency due to the energy transfer with the target molecules. When excited at a specific frequency, the target molecules can either increase or decrease in vibrational energy. Thus, the small fraction of the scattering light reduces (Stokes shift) or gains (anti-Stokes shift) equally the energy of the molecule vibration.

Due to the unique covalent bonds and an atomic mass of each molecule, different molecules require specific excitation energy to change the molecule vibration. The combination of multiple energy shifts creates a unique spectrum for each target molecule. The distinct spectra can be used to identify the target molecule in Raman spectroscopy.

Contrast agents in surface-enhanced Raman spectroscopy (SERS) can be used to amplify the Raman spectra of the target samples. As the electromagnetic wave (laser) irradiates the contrast agent molecules, it excites the localized surface plasmons on the rough surface. This results in the enhancement of electromagnetic fields near the surface. The increase in the intensity of the electromagnetic fields would also increase the intensity of Raman scattering and thus amplifies the Raman spectra. As a result, by coupling the contrast agents with the target molecules, the SERS technique can be applied for identification of target molecules.

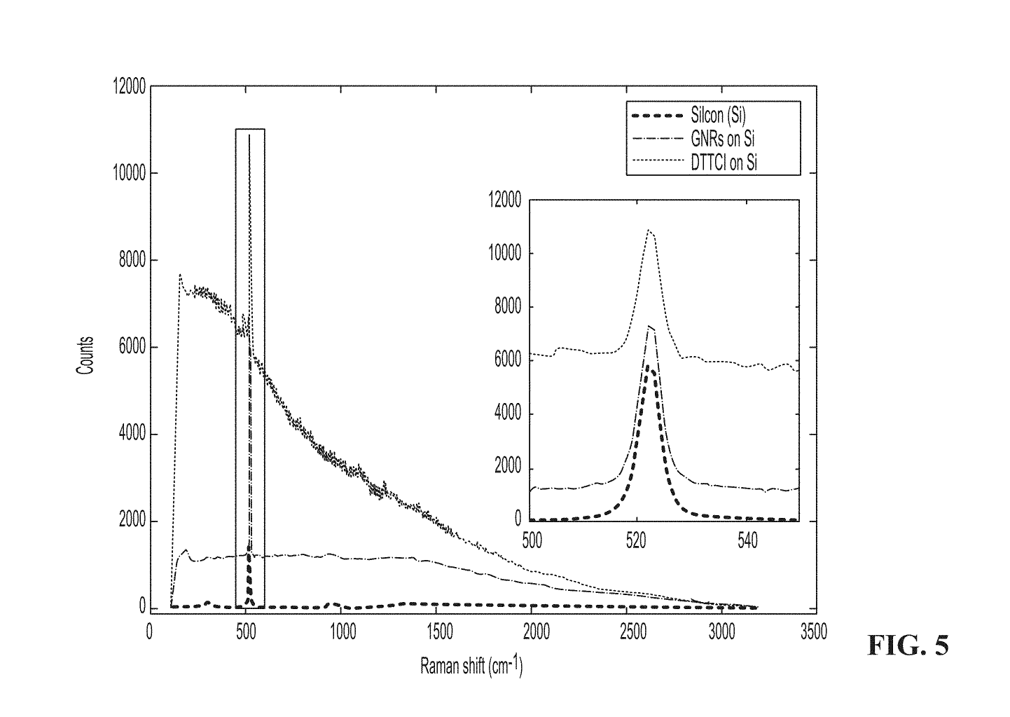

Gold nanorods (GNRs) (Sigma Aldrich) and 3,3′-Diethylthiatricarbocyanine iodide (DTTCI) (Sigma Aldrich) were used as the contrast agents in SERS detections to verify the material of the 3D printed object. The contrast agent can be embedded in the filament at specific locations for material identification. The internal structure of the 3D printed object can be verified using the embedded materials. FIG. 5 shows the result of the standard Raman scattering measurement of the Silicon (Si) wafer and the Raman scattering of GNRs and DTTCI drop coat on top of the wafer. The Si wafer was used to calibrate the Raman instrument prior to the experiments. The Si Raman spectra have been studied thoroughly. As shown in FIG. 5, the GNRs and the DTTCI amplified the signal response of the Si Raman scattering intensity.

### Computed Tomography

The second scanning modality is a computed tomography (CT) scan. Just as in the SERS experiment, it is necessary to find an effective contrast agent that would allow us to view the embedded materials within the 3D printed model. GNRs was again used because gold works as an excellent contrast agent due to its X-ray density. In addition, the biocompatibility of GNRs is beneficial for the verification procedures for the tibial prosthesis.

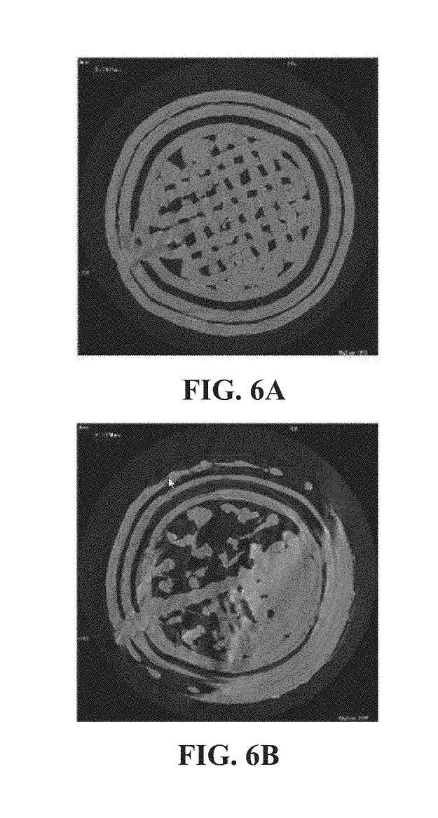

It was initially experimented with the use of GNRs as a contrast agent for CT scanning by embedding them in a simple 3D printed model. A cylindrical 3D model was developed and printed using standard acrylonitrile butadiene styrene (ABS) filament as the control material of the model. Multiple layers of ABS filament with embedded GNRs were deposited in between the bulk material. FIG. 6A and FIG. 6B shows the initial results of the 3D printed model with a layer of injected GNR filament. FIG. 6A shows an ABS control print, and FIG. 6B shows a GNR layer print. CT scan was performed using a Skyscan 1172 microCT scanner. As shown in FIG. 6, the result demonstrated that the GNRs could be a contrast agent for the material verification.

### Example 2

Evaluation

In this section, the three-layered verification method was evaluated. The identification of a malicious print, the observation of the detected error, and the post-production materials verification are described. Then, the effectiveness of the acoustic and spatial verification on the use case of a 3D printed tibial knee implant was evaluated.

To quantify the accuracy of the results of the various tests, the data were fit into a logistic regression model with the binary dependent variable of “malicious print detected” or “no malicious print detected.” From the model, the probabilistic classification outcomes were extracted to create a receiver operating characteristic (ROC) curve. The area under the ROC curve (AUROC) is the metric used to define classification accuracy.

### 2.1. Identification of Malicious Prints

In this section, the effectiveness of the proposed verification method was evaluated in simply identifying an error in a potentially malicious print. This initial identification was carried out primarily by the acoustic layer with redundancy in the spatial layer to reduce false classifications.

### 2.2. Classification Accuracy

To gain an initial understanding of the parameters that affect the accuracy of the acoustic layer, several experiments were carried out with a small number of trials. The printers used in the tests were a Lulzbot Taz6, Lulzbot TazMini, and an Orion Delta. The AKG P170 condenser microphone was placed on a stand as close to the moving extruder head without being knocked over by the moving components of the printer. The audio classifier is called Dejavu and is an open-source project written in Python (Drevo Will. Dejavu; https://github.com/worldveil/dejavu (2017)).

To generate data useful for logistic regression, a vector of scores, S, is generated using the method described above. For example, the components of S are shown in FIG. 3. The vector S is of length n, where n=[audio length/audio slice length]. A print score, p, is then calculated, where:

$$p = {\sum }_{n} {S}_{n} \qquad ( 1 )$$

The value p associated with a given print determines how likely the print is to be the same as the training print, with higher values meaning more likely and lower values meaning less likely.

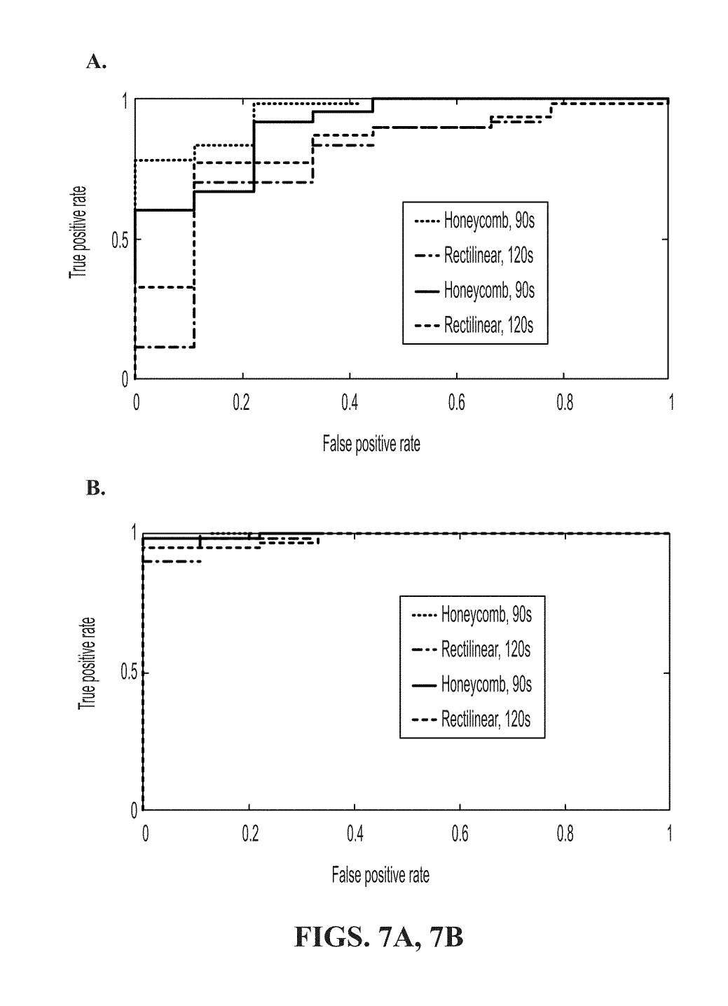

FIG. 7A shows the ROC curves for the classification results of honeycomb and rectilinear fills with the audio segmented to 90-second and 120-second segments (each CTh=35). The same original audio files were used whether the audio files were segmented to 90-second segments or 120-second segments. The honeycomb and rectilinear tests each consist of nine target prints and sixty malicious prints. The reason for a large number of known positive error classifications was that each print is considered an erroneous version of each other print.

The poorest performance was 78.15% accuracy for the rectilinear fill with the audio segmented at 90 seconds. Such an accuracy is unacceptable, especially considering the high likelihood of false positives. To find an explanation for the poor classification, the G-code was inspected. Upon investigation of the G-code which was generated by Slic3r, it was found that 9 lines which specified x and y coordinates along with the extrusion rate were repeated 12 times each out of 15 layers needed to complete the print in both the rectilinear and honeycomb fill patterns. Also, upon investigating sequentially repeated blocks of code, blocks of G-code describing three entire layers were repeated twice during the course of the print. This symmetry was hypothesized to be the cause of the classification confusion.

To test this hypothesis, a second set of tests were conducted with a top hat design (FIG. 2A), which is asymmetrical along the z-axis. The same number of prints was performed with honeycomb and rectilinear fill being sliced to 90 seconds and 120 seconds each and CTh set to 35. The ROC curve of these experiments is shown in FIG. 7B. Each sample consists of nine target prints and sixty malicious prints, and the same data were used for the 90-second audio slice length as the 120-second slice.

Upon investigating the G-code, the only repeated lines were those that define the nozzle speed at the beginning and do not include extrusion. Furthermore, there are no blocks of G-code or layers that are entirely repeated verbatim. This is suspected to contribute greatly to the increased performance seen in FIG. 7B. The least accuracy is 98.52%, which is suitable for verification purposes. Between the 120-second and 90-second slice lengths, little change in performance was observed. Although audio classification is effective in identifying malicious prints, it is still susceptible to both false positives. By introducing data from the spatial layer, false positives may be reduced. For instance, FIG. 7C compares the data from the x, y, and z axes of the 40% honeycomb and 40% rectilinear fills from FIG. 4. A significant difference between the two prints was identified. Each frequency response has a similar shape, but the major features of the 40% rectilinear fill are shifted to the right because the back-and-forth motion is not impeded by the creation of small honeycomb structures.

For classification, the four most prominent peaks were used as features along with their locations. A test was conducted in which the target print was chosen to be the 20% rectilinear disk shown above. All other prints were considered malicious. As a result, 10 target prints and 12 malicious prints were obtained. When trained using the linear regression model, 100% accuracy was achieved in differentiating between malicious and target prints.

While the spatial sensing layer is primarily for the purpose of print visualization, its role in conjunction with the acoustic layer allows for 100% accuracy in detecting malicious prints.

### Varied Printer Models

To understand the effectiveness of audio classification for print verification on different printer models, several prints were performed on a LulzBot Taz Mini and an Orion Delta, respectively. Acoustic data recordings were obtained using the same microphone. In each print, a top hat design identical to the one described above was printed, and the audio was sliced to 120 s. The optimized CTh for the Taz Mini, Orion Delta, and Taz6 are 150, 20, and 35 respectively. The ROC curve results are shown in FIG. 7D. Because the honeycomb and rectilinear fill patterns are considered together, each data set consists of 18 target prints and 120 malicious prints. Thus, the acoustic verification method is generalizable to printers of different sizes and configurations. The classification accuracy did not fall below 95.42% in these tests.

### Classification in Noisy Environments

Other experiments were conducted using an Afina H40 3D Printer with an eBoTrade Digital Voice Recorder wide-range microphone. This setup was in a noisy university makerspace with people talking near the printer. In this experiment, the classification accuracy suffered greatly (near 50% accuracy). Although acoustic verification is useful on different types of printers above, the loss of classification accuracy was likely due to the noise in the environment. Also, because the microphone was not directional, the noise near the printer can be heard by the microphone as well. Therefore, in implementing this verification scheme, higher classification accuracy would be achieved by using directional microphones and noise isolation.

### 2.3. Visualization of Malicious Prints

When a potentially malicious print is identified, it is important to have the capability to visualize the potential threat. The visualization must be independent of the intended G-code which may be interpreted differently by malicious firmware. This is achieved in real time through the use of the spatial sensing layer and in post-production by the materials inspection layer.

### Real-Time Visualization

In the event that a potential malicious print is identified, a user has the capability of viewing the real-time print in progress through the spatial sensing as seen in FIG. 4. By viewing the layer in progress, significant fill pattern changes such as those between the 20% honeycomb and 20% rectilinear fill are obvious. However, less obvious changes made to the print such as those between the 40% honeycomb and rectilinear fills could be identified through FFT analysis (FIG. 7C). This is particularly true if the user has access to the frequency response of a reference print.

While the spatial sensing layer is useful for identifying the type of fill pattern that is being maliciously generated, it is less useful for identifying if the design itself has been altered due to the warping that occurs in the data. This, however, is an easy issue to solve through the use of a webcam which can easily identify the shape of the design. In this sense, it may seem that spatial sensing may be replaced altogether by a webcam, but it is important that the latter uses far more data and does not readily provide information about the frequency response.

### Post Production Visualization

The aforementioned materials-based verification methods were meant to be generalized for any scanning method that can detect the embedded contrast material within a 3D model. In this study, Raman spectroscopy and computed tomography were used because these modalities were readily available to us at the time of evaluation.

As shown in FIG. 5, 10 nm diameter GNRs 780 nm absorption and DTTCI 765 nm absorption (Sigma Aldrich) diluted in ethanol were used as the two distinct contrast agents. Each contrast agent was drop coated on the surface of the 3D printed disk. The Raman spectra of the blank 3D printed disk were also taken as the control data. The results in FIG. 5 show that the GNRs and DTTCI can be combined for use as a contrast agent in Raman spectroscopy.

To emulate the filament with the embedded contrast agent, the filament from ABS pellets was produced using the filament maker (Filabot). For the GNRs embedded filament, the ABS pellets were submerged in a GNR solution and left to dry. In this test, a 4 mL GNR solution was mixed with 12 g of pellets. Based on the information from the manufacturer, the number of GNRs per mL of solution was calculated to be approximately 7.284e11. Per 12 g of pellets, approximately 2 m of a filament with a 2.5 mm diameter can be produced. The 3D printed disk has 50 μm in layer thickness. Therefore, for the area of 1 μm² on each layer of the 3D printed disk, there are approximately 4 GNRs particles. This approximation only serves as the estimation of the GNRs within the measurement area. Due to the non-uniform mixing of the GNRs in the pellets, the distribution of GNRs within the 3D printed disk varies considerably. For the DTTCI embedded filament, while the quantity of DTTCI in the filament was not estimated, larger quantities of the DTTCI enhancer were available to produce the modified filament. The blank ABS filament was extruded using only ABS pellets.

### Precise Embedding of Contrast Agents

The contrast agents or markers may be embedded at precise Cartesian coordinates within the 3D printed models. For example, a Lulzbot Taz dual extruder tool head can be used to provide the capability of localizing the embedded filament at precise locations. However, to demonstrate the concept, ABS filament was saturated in the GNRs or DTTCI throughout the entire spool of filament.

In the following subsection, the Raman spectra of the blank 3D printed disk were evaluated, the 3D disk with GNRs or DTTCI drop coat on the surface, and the 3D printed disk with GNRs or DTTCI embedded filament. The G-code may be modified to allow the user to embed filament at desired locations, i.e., switching/alternating between the extruder nozzle containing the normal filament and the nozzle containing the GNR filament. The user can specify the beginning and end points of embedded material within the normal print path. This method was used for both the initial CT scan results as well as the final evaluation.

### Imaging Analysis.

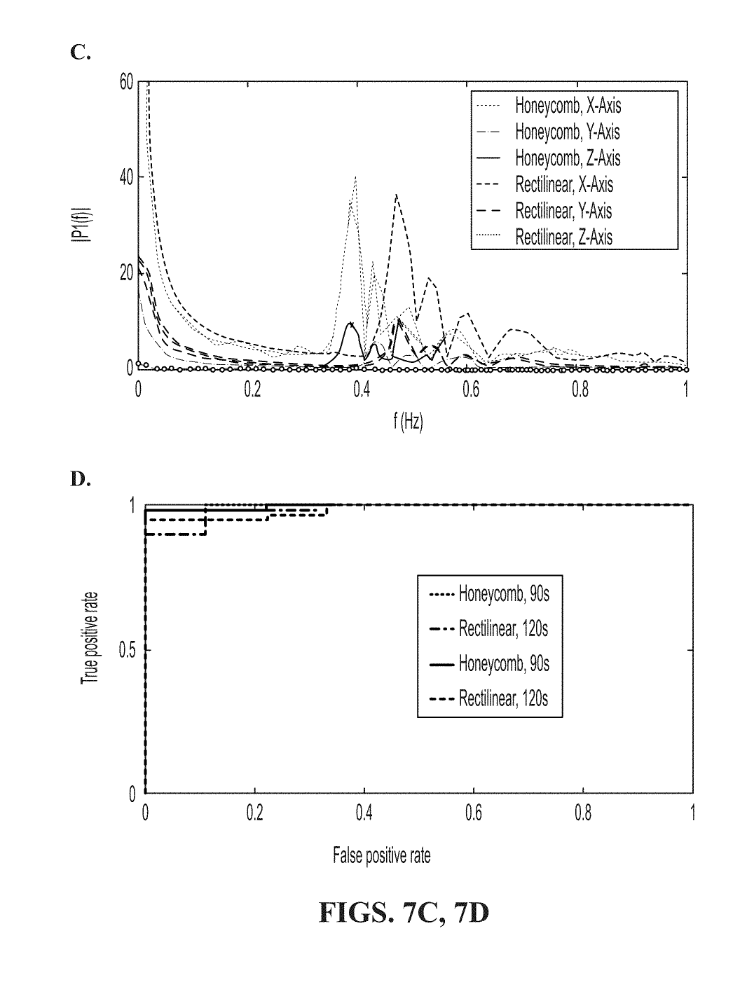

In the evaluation using Raman spectroscopy, the 3D printed disk was excited with 785 nm infrared light for 20 seconds per accumulation of data at 100% power setting in Renishaw InVia micro-Raman system. FIG. 8 shows the mean measurement results all data spectra of the 3D printed disks. Similar to the results from FIG. 5, the spectrum of the 3D printed disk with DTTCI coated surface has significant improvement of photons counts across the spectrum comparing to the control data of the blank 3D printed disk. The spectra of the 3D printed disk from DTTCI embedded filament also shows the elevation of photons counts comparing to the control data. These spectra fall in between the spectra of the control data and the surface coated 3D printed disk. This conforms with the fact that the surface coated would accumulate more contrast agent at the measurement site comparing to the embedded filament. While the Raman spectroscopy can be used to quantify the concentration of the target particles, the elevation of the photons count in FIG. 8 does not reflect the approximate distribution of the contrast agent embedded in the filament. The measurement site in Raman spectroscopy might be a cluster or spare of contrast agent or markers. As mentioned earlier, the markers might not be uniformly distributed in the filament. The high reflection in the CT scan shows the large cluster of the GNRs in the embedded filament. Due to the low resolution of the micro CT scanner, the scan would not highlight the areas where the GNRs are sparsely distributed. While the Raman spectroscopy results of the GNRs embedded filament are not shown, the similar response can be discerned.

In the classification of 3D printed blank ABS, GNRs embedded, and DTTCI embedded disk, mean and standard deviation of the spectra were used to distinguish the cluster of data set. FIG. 8 shows the mean of the typical response of Raman spectra of the 3D printed disk with blank ABS, DTTCI coated disk, and DTTCI embedded ABS filament. The greatest change of Raman shift was in the range of 100 cm⁻¹ and 800 cm⁻¹. The Raman scattering separation was within the range of 791.21 nm and 837.60 nm scattering, when the sample was irradiated at 785 nm. Therefore, this is the reasonable range of interest for Raman scattering for all data selection. By training the logistic regression model, the classification using mean and standard deviation shows 100% accuracy against the blank ABS (226 samples) filament for both GNRs (179 samples) and DTTCI (71 samples) embedded filaments.

In Raman spectroscopy, the maximum setting depth penetration for the Renishaw InVia micro-Raman system is approximately 300 μm, the 3D printed object where the GNRs or DTTCI embedded filament was implanted further inside the object was not verified. Therefore, the Raman spectroscopy would not be sufficient for the verification that requires depth. In further analysis, the micro CT scanner was used to evaluate the internal structure of printed 3D objects.

The results for the CT scan approach presented in FIG. 6 showed that although the GNRs embedded filament contrasted well in the CT scan, the custom filament cannot be used due to the sparse distribution of the GNRs. As an alternative, commercially available stainless steel filaments were used where the filament is heavily saturated with stainless steel particles. Under the CT scanning, the steel particles would produce a similar response to the GNRs due to high X-ray density. Although stainless steel is not biocompatible, it will serve as a substitute for the GNRs to provide precise visibility in the CT scan. Furthermore, the control filament is changed from ABS to polylactic acid (PLA) after comparing the densities in the CT scan. The X-ray properties of PLA versus ABS have been studied, our assumption was confirmed after simple trial and error. Evaluation of this approach on a tibial prosthesis will be discussed in the subsequent section.

### Example 3

### Case Study: Prosthetic Knee

In the case study described below, the tibial implant portion of a prosthetic knee was used. Unlike the titanium alloy component of the prosthetic knee that attaches to the femur, the tibial portion of the implant is made from polyethylene and has been identified as a component that could easily be manufactured through AM. Furthermore, the knee undergoes more mechanical stress than any joint. Thus much research has been conducted which describes the medical implications of its wear and tear. Therefore, an attack is considered in which alterations are made to the internal structure of tibial knee implant would dramatically increase the rate of wear.

As described above, a model of the tibial component of a prosthetic knee implant was used as a design for a use case test. Prosthetics differ slightly between patients, so it is assumed that malicious print identification is performed periodically with a known standard prosthetic design. Real-time and post-production visualization were still performed on each print.

### Error Identification

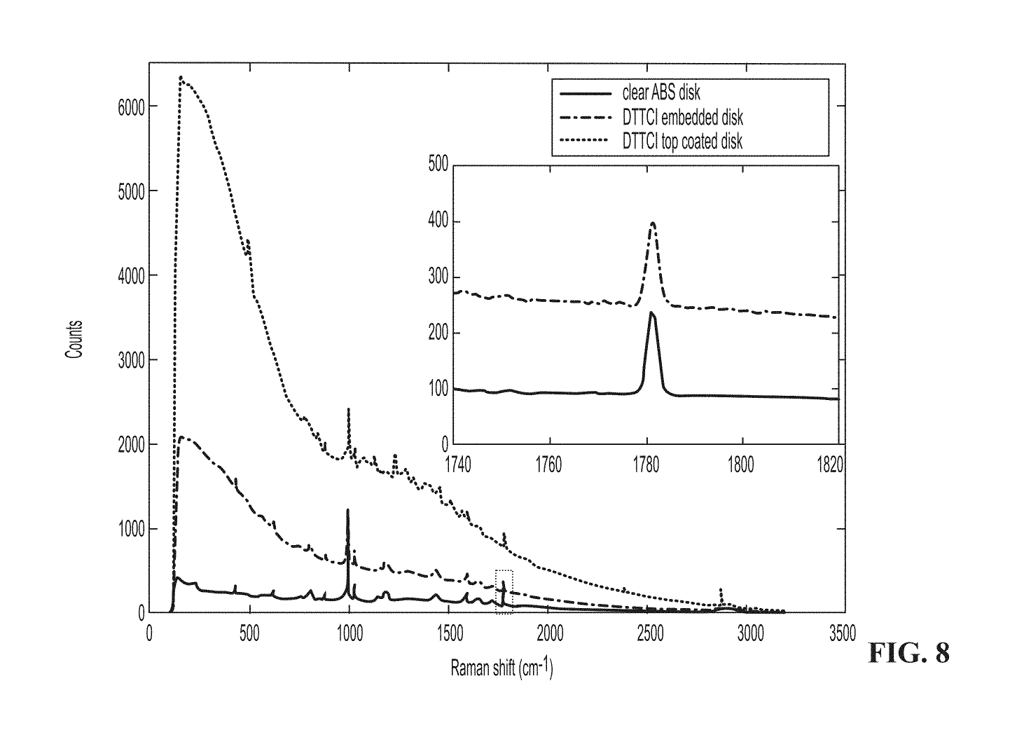

FIG. 9 shows the confidence values of the acoustic verification for both the target print and the malicious print. These results were gathered using the same technique as those described in section 3 with audio slices of length 120s and CTh=0. By setting CTh=0, a positive error classification can be made within the first 360 s of the print or the first 4% of the total known print time by only observing out-of-sequence index classifications. The CTh may be set to anything less than 18 without causing a false positive. Overall, acoustic error detection itself saves over 2 hours of print time and prevents a potentially harmful print from being completed.

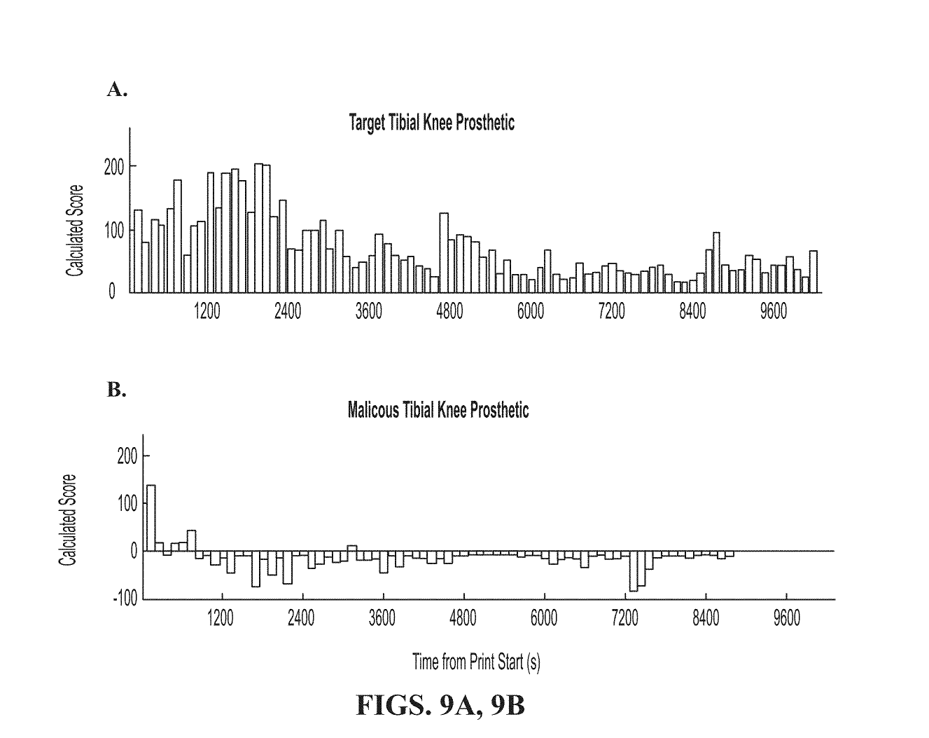

In FIG. 10, the FFT analysis of a target print and a malicious print were compared to a training print. Similar to FIG. 7C, the malicious print shows a different frequency response near 0.2 Hz as highlighted by the lower box. The upper box highlights the closeness of the peaks between the training and target prints and the difference between those and the malicious print. The full print of the object requires 111 layers, so it would take less 1% of the time of the total print to identify the erroneous pattern once it begins.

### Real-Time Visualization

In this test, the target print uses a 60% rectilinear fill and the malicious print uses a 20% honeycomb fill. In the attack, the visualization of the intended G-code remains unaltered for the user while the instructions sent to the printer were altered. The consequences of this attack would be to cause accelerated wear in the implant causing pain and financial loss for the victim who has the implant.

For the print identification and real-time visualization tests, a full-sized prosthetic design was used. However, due to the size limitations of the MicroCT scanner, a significantly scaled down version of the same design was used.

The training, target, and attack prints were each recorded on the Lulzbot Taz6 printer. Due to the availability of the experimental setup, a single layer of each of these prints was performed by the Dobot Magician for the visualization tests. The same G-code was used, or the Dobot prints as in the Taz6 with the exception of the extruder being disabled and the speeds decreased to suit the capabilities of Dobot. Spatial verification testing can be performed on the Taz6, because it measures the relative position between the nozzle and the base, regardless of whether that base is a stationary table or a moving part of the printer. Also, acoustic and spatial verification may be performed in concurrently or sequentially (as in this study).

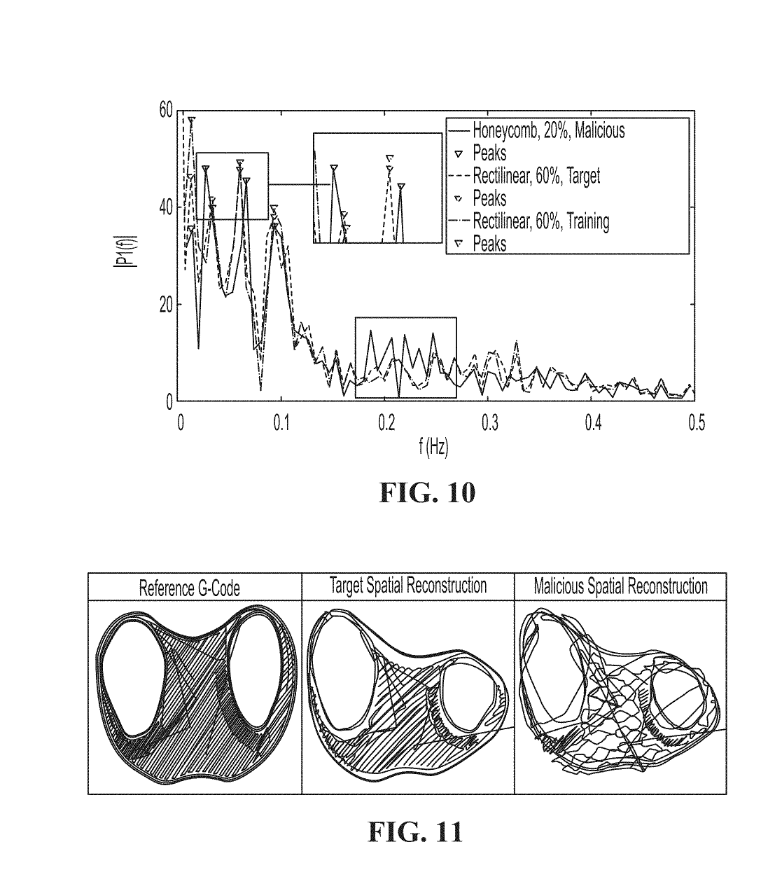

FIG. 11 shows the spatial verification visualization of, in order of left to right, a G-code visualization of the training print, a spatial reconstruction of the target print, and a spatial reconstruction of the malicious print. It is clear that the recreated target print uses a rectilinear fill at approximately the correct density while the malicious print differs significantly from the intended G-code. Due to the warping that occurs in the spatial reconstruction, a user would not be made aware if the shape of the print were altered by using this method alone.

### Post Production Visualization

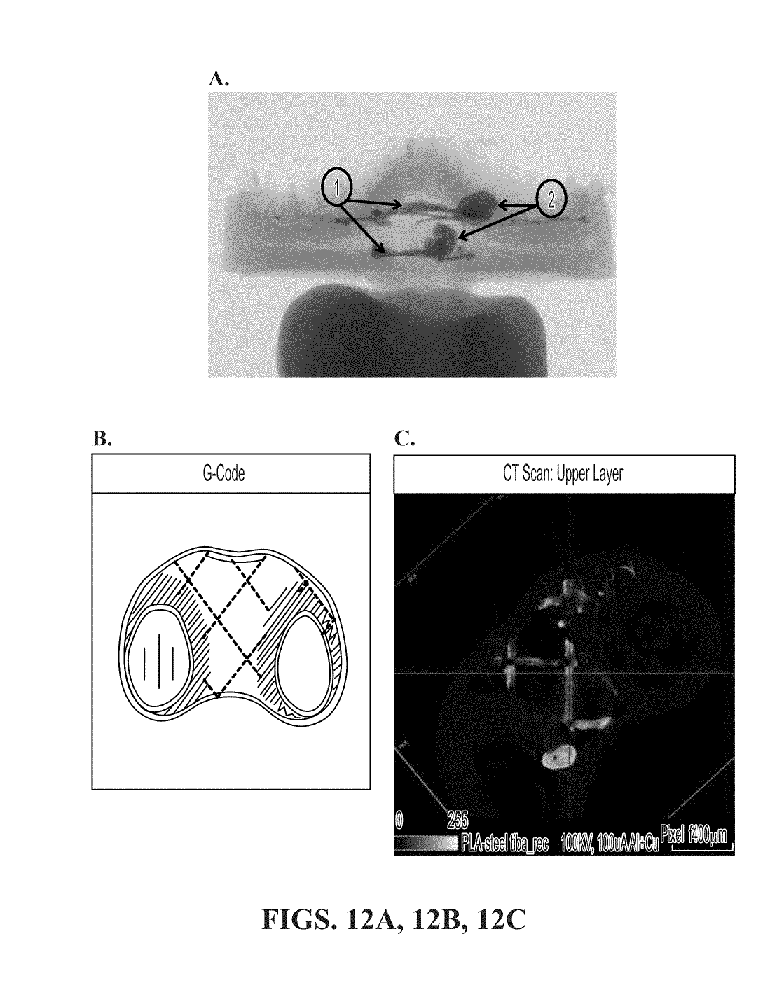

The CT scan approach is preferred for the post-production visualization, as the Raman spectroscopy would not be able to verify the internal structure of the tibial prosthesis due to its depth limitations. FIG. 12A shows an X-ray scan of the front of a PLA tibial prosthesis with 2 infill layers of steel. Because only a microCT scanner is available for use, the part of the tibial insert was scaled down to fit within a diameter of about 30 mm. The two large blotches of stainless steel are simple imperfections that mark the point where the second extruder began printing.

FIG. 12A and FIG. 12B compares the intended print of the top stainless steel layer—with the stainless steel path shown as dotted-lines-versus the CT scan of that layer at a 15 am/voxel resolution. The CT scan image is rotated about 45 degrees in comparison to the intended print. Furthermore, the small model had to be mounted on a bed of silicone polymer to hold it in place, so it is not completely level. Despite the imperfections of the printed model and the scans, it can be seen that the steel was properly embedded within the walls of the model and is clearly detectable against the PLA filament.

The use of the word “a” or “an”, when used in conjunction with the term “comprising” in the claims and/or the specification, may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features, and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

We claim:

1. A method for verifying the integrity of an additive manufacturing process, comprising:
printing a three-dimensional (3D) object using a filament, wherein the filament comprises a first material and a second material, wherein the second material is embedded in the first material in a predetermined pattern;
determining a reference location of the second material in the 3D object based on the predetermined pattern of the second material in the filament;
detecting a target location of the second material in the 3D object;
calculating a difference between the reference location and the target location; and
determining the presence of alteration of the additive manufacturing process if the difference between the reference location and the target location is above a threshold value.

2. The method of claim 1, wherein the second material comprises a nanoparticle.

3. The method of claim 1, wherein the second material comprises a gold nanorod.

4. The method of claim 1, wherein the second material comprises 3, 3′-Diethylthiatricarbocyanine iodide.

5. The method of claim 1, wherein:
the predetermined pattern is encoded in a barcode; and
determining the reference location of the second material inside the 3D object is based on decoding the barcode to extract the predetermined pattern of the second material.

6. The method of claim 1, wherein the step of detecting the target location of the second material in the 3D object further comprises detecting the target location of the second material in the object using an imaging tool selected from the group consisting of an X-ray, an IR, a Raman, and a computed tomography.

7. A method for verifying the integrity of an additive manufacturing process, comprising:
recording, by an audio sensor, an audio signal generated by the 3D printer during the performance of the 3D print job;
comparing, by a computer, the recorded audio signal with a reference recording during or after the performance of the 3D print job;
determining a difference between the recorded audio signal and the reference recording; and
determining that the additive manufacturing process is altered if the difference is below a threshold value.

8. The method of claim 7, wherein the threshold value is the confidence threshold (CTh).

9. The method of claim 7, wherein each of the audio signal and the reference recording comprises a plurality of audio segments.

10. The method of claim 9, wherein the step of comparing the audio signal with the reference recording further comprises determining a difference between an audio segment of the audio and a corresponding audio segment of the reference recording.

11. The method of claim 10, further comprising determining that the additive manufacturing process is altered if the difference is below the threshold value.

12. The method of claim 8, wherein the plurality of audio segments comprises a 90-second or 120-second audio segment.

13. The method of claim 7, wherein the audio sensor is a directional microphone.

14. A method for verifying the integrity of an additive manufacturing process, comprising:
recording, by a sensor, a trajectory of a printer head of a three-dimensional (3D) printer during a performance of a 3D print job; and
determining a presence of alteration of an additive manufacturing process associated with the performance of the 3D print job based on the recorded trajectory.

15. The method of claim 14, wherein the step of determining the presence of alteration further comprises:
comparing, by a computer, the recorded trajectory with a reference recording during or after the performance of the 3D print job; and
determining the presence of the alteration of an additive manufacturing process associated with the performance of the 3D print job based on a difference between the recording trajectory and the reference recording.

16. The method of claim 14, further comprising:
generating instructions for causing the printer head to produce a signature trajectory during the performance of the 3D print job,
wherein the step of determining the presence of the alteration comprises:
determining, by the computer, whether the signature trajectory is present in the recorded trajectory; and
determining that the additive manufacturing process associated with the performance of the 3D print job is altered if the signature trajectory is absent in the recorded trajectory,

17. The method of claim 14, wherein the sensor comprises at least one of a gyroscopic sensor and a linear potentiometer.

18. The method of claim 14, wherein the sensor is mounted on the printer head of the 3D printer.
